(19) United States
(12) Patent Application Publication
(10) Pub. No.: US 2022/0280330 A1
(43) Pub. Date: Sep. 8, 2022

(54) Penile straightening devices

(71) Applicant: Stephen Albert Osella, AUSTIN, TX (US)

(72) Inventor: Stephen Albert Osella, AUSTIN, TX (US)

(21) Appl. No.: 17/305,080

(22) Filed: Jun. 30, 2021

Related U.S. Application Data
(60) Provisional application No. 63/156,723, filed on Mar. 4, 2021.

(51) Int. Cl. A61F 5/41

(52) U.S. Cl. CPC A61F 5/41; A61F 2005/411

(57) ABSTRACT

Penile straightening devices are disclosed. The proposed invention is used to passively or actively straighten or counter the curvature of an erect penis to reduce or completely eliminate the curvature in a painless, inexpensive, and safe manner.

### BACKGROUND

Curvature of a fully or semi-erect penis resulting from, but not limited to, Peyronie's Disease, has been linked to erectile dysfunction, pain during intercourse, and psychological issues such as depression. The curvature of the erect penis when suffering from Peyronie's Disease is caused by the presence of plaque somewhere in the penis that constrains the expansion of the penis around the area of the plaque during an erection. When the penis expands during the course of achieving an erection, the area around the plaque does not expand completely, thus causing a curvature of the penis.

The reasons for the plaque formation in Peyronie's Disease are not fully understood at present time. Early studies estimated the average incidence of Peyronie's Disease to be between 1% and 3%; however, newer studies are estimating occurrence may be closer to 10%, with a steep age-related increase over the age of 40.

The existence of Peyronie's Disease has been chronicled since at least the 13^{th} Century. The only treatment for Peyronie's Disease in the past had been surgery. As of the year 2021, approaches for the treatment of Peyronie's Disease include medical procedures, such as surgical removal and Xiaflex® (Collagenase Clostridium Histolyticum), traction devices, manual traction, heat therapy, and Electro Shock Wave Therapy (ESWT). These approaches are either ineffectual, painful, costly, and/or potentially dangerous.

The proposed invention specifically targets the treatment of penile curvature by constraining or countering the curvature of the penis while the penis is erect. The proposed invention is to be used when the penis becomes 1) erect naturally (spontaneous erection), or 2) artificially through the use of erection methods and devices, such as Vacuum Erection Devices (VEDs). When the proposed device is worn while the penis is erect, the device keeps the erect penis straight, or bends the penis in the opposite direction of the curvature. Through repeated use of the proposed invention, the plaque will be altered such that the penile curvature will be reduced or eliminated.

The proposed invention can be used either in a static configuration, or with the active manipulation by the patient to counter the curvature. When used in conjunction with artificial erection devices, the proposed invention will be used in a static configuration, either straight or with a bend opposite to the curvature. Active manipulation is also referred to as modeling. Modeling can be performed by the user with the proposed invention in and of itself, or in conjunction with medical treatments such as, for example, but not limited to, Xiaflex® (Collagenase Clostridium Histolyticum). When used in conjunction with medical treatments, the proposed invention enhances the efficacy of the manual modeling, which is typically prescribed during treatment.

### SUMMARY

The proposed invention is specifically targeted to treat the curvature of an erect penis, the curvature of which could have been caused by, for example, but not limited to, Peyronie's Disease. The penile curvature is only evident when the penis is erect or semi-erect. In the case of Peyronie's Disease, the curvature is caused by the inhibition of penile expansion during erection around the area of a plaque that has built-up somewhere in the penis.

When in use, the proposed invention counters the curvature of the erect penis by keeping the penis either straight, or at an angle opposite to the curvature, depending on the configuration of the proposed invention (see Drawings), or by active manipulation by the user. Active manipulation, referred to as modeling, acts upon the plaque such that, when the proposed invention is not used, the erect penis will not exhibit the extent of the curvature as it did prior to treatment using the proposed invention. The extent of the reduction of the curvature lessens with the continued use of the proposed invention.

In addition, at the onset of the disease, if the erect penis is forced to straighten, due to intercourse for example, the patient generally feels pain and discomfort since the plaque is constraining the penile expansion. The proposed invention gradually results in a reduction or elimination of pain as the plaque, and the area of the penis around the plaque are permanently stretched.

The proposed invention can be used by itself to perform manual modeling, or in conjunction with artificial erection devices such as, but not limited to, Vacuum Erection Devices (VEDs). The proposed invention is worn during the time the penis becomes and remains erect. While erect, the penis is kept straight, or bent in the opposite direction of the curvature, depending on the configuration, or by active manipulation by the user performing manual modeling.

In accordance with one embodiment, a penile straightening device comprises a non-bendable tube with rounded or flanged edges, and optionally an inner-tube liner. In accordance with another embodiment, the penile straightening device comprises a curved non-bendable tube with rounded or flanged edges, and optionally an inner-tube liner. In accordance with another embodiment, the penile straightening device comprises two or more non-bendable tubes with rounded or flanged edges that are connected with latching mechanisms that allow the tubes to be configured at desired angles and locked into place, and optionally an inner-tube liner.

### DESCRIPTION OF THE DRAWINGS

The proposed invention is envisioned in three configurations, which are illustrated in the drawings contained in this application.

FIG. 1 depicts the simplest configuration of the proposed invention. As can be seen in FIG. 1, the proposed invention in this configuration is a non-bendable tube composed of a hard material. The flaccid, or semi-erect penis enters one side of the non-bendable tube, and is positioned inside the non-bendable tube such that the middle of the location of the curvature is fully contained within the non-bendable tube, preferably at the middle of the non-bendable tube. It is often the case that the precise location of the curvature, and thus the location of the plaque, is very apparent. The edges of the non-bendable tube are rounded and/or flanged so as to not injure the penis during use. Optionally, there is an inner-tube liner, also so as to not result in injury.

FIG. 2 depicts a slightly more complex configuration of the proposed invention. As can be seen in FIG. 2, similar to the configuration depicted in FIG. 1, this more complex configuration also consists of a single non-bendable tube composed of a hard material. However, in the configuration depicted in FIG. 2, rather than being completely straight as in FIG. 1, the tube is curved. The degree of the curvature of the tube can vary depending on the curvature of the penis, or medical or personal reasons. In this configuration, the penis also enters the tube at one end, and the other end of the tube is positioned such that the rounding of the tube is positioned opposite to the curvature of the penis. Thus, when the penis becomes erect, the countering of the curvature is even more pronounced than achieved using the configuration depicted in FIG. 1. As with the previous configuration, the edges of the non-bendable tube are rounded and/or flanged so as to not injure the penis during use. Optionally, there is an inner-tube liner, also so as to not result in injury.

FIG. 3 depicts the most complex configuration of the proposed invention. In this configuration, the tube is divided into two or more non-bendable tube sections, each of which can be positioned at different angles. The sections of the non-bendable tubes are kept in place at the desired angle by a latching mechanism that connects and fixes the sections of the non-bendable tubes. This configuration provides the greatest flexibility of design, but is the most complex to manufacture and use. As with the previous configurations, the edges of the non-bendable tube are rounded and/or flanged so as to not injure the penis during use. Optionally, there is an inner-tube liner, also so as to not result in injury.

For all configurations, the diameter of the tubes to use will depend on the thickness of the patient's erect penis. The key is that the tube should feel snug around the patient's erect penis without being too tight. All that is required is that penis is kept straight, or in a position where the curvature of the tube is opposite to that when the penis is erect when not using the proposed invention.

### DETAILED DESCRIPTION

The proposed invention is specifically targeted to treat the curvature of an erect penis that has been caused by, for example, but not limited to, Peyronie's Disease. The curvature occurs in an erect or semi-erect penis around the area of plaque that has built-up as a result of the disease. The plaque inhibits the expansion of the penis around the area of the plaque while the remainder of the penis expands normally. It is important to understand that the curvature of the penis is only experienced when the penis is fully or semi-erect, that is, when it has expanded from its usual flaccid state.

The proposed invention can be used by itself, or in conjunction with the function of artificial erection devices such as a Vacuum Erection Device (VED), which is to bring blood into the penis to achieve an erection. The proposed invention 1) counters the curvature, thus stretching the plaque causing a break up of plaque (micro fractures), and 2) allows the blood that is brought into the penis by the erection device to enter the plaque to enable healing (recomposition) of the plaque. It is this dual function that makes the proposed invention more effective than artificial erection devices, or traction, by themselves.

The claimed invention differs and is an improvement from what currently exists. The main methods for treating penile curvature are medical procedures such as surgical removal and Xiaflex® (Collagenase Clostridium Histolyticum), traction devices, manual modeling (traction), Electro Shock Wave Therapy (ESWT), and artificial erection devices such as Vacuum Erection Devices (VEDs). Medical procedures have been shown to be effective, but are very painful, costly, and dangerous. Traction devices have not shown to be much more effective than manual modeling, and in some cases have worsened the condition. Electro Shock Wave Therapy (ESWT) has not been shown to be effective. The proposed invention is much less painful, less costly, and much safer than existing methods.

### Versions of the Invention and Their Components

1. Single Straight Non-Bendable Tube
a. Single straight non-bendable hard tube
b. Rounded or flanged tube edges
c. Optional inner-tube liner

2. Single Curved Non-Bendable Tube
a. Single curved non-bendable tube
b. Rounded or flanged tube edges
c. Optional inner-tube liner

3. Non-Bendable Tubes With Adjustable Angles
a. 2 or more sections of non-bendable tubes
b. Rounded or flanged tube edges
c. Optional inner-tube liner
d. 1 or more latching mechanisms

### Relationship Between the Components

### 1. Single Straight Non-Bendable Tube

The flaccid penis enters Item #1.a and, when erect, can remain completely with Item #1.a, or slightly exit Item #1.a with the location of the plaque (curved section of the penis when erect) remaining fully within Item #1.a. The tubes have rounded or flanged edges, Item #1.b, and, optionally, an inner-tube liner, Item #1.c.

### 2. Single Curved Non-Bendable Tube

The flaccid penis enters Item #1.a and, when erect, can stay completely with Item #2.a, or slightly exit Item #2.a with the location of the plaque (curved section of the penis when erect) remaining fully within Item #2.a. The location of the plaque is place at the center of Item #2.a, and the curvature of Item #2.a is positioned opposite to the curvature of the erect penis. For example, if the curvature of the erect penis is upwards (dorsal location of the plaque), the curvature of Item #2.a is directed downwards. The tubes have rounded or flanged edges, Item #2.b, and, optionally, an inner-tube liner, Item #2.c.

### 3. Non-Bendable Tubes With Adjustable Angles

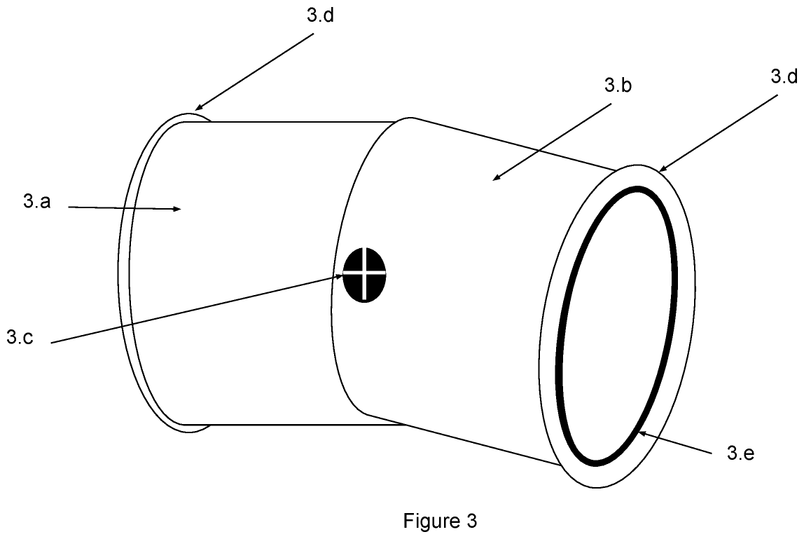

FIG. 3 depicts 2 non-bendable tube sections; however, configurations can consist of more than 2 sections. The flaccid penis enters Item #3.a and continues into Item #3.b. The penis, when erect, can remain entirely within Item #3.b, or can slightly exit Item #3.b. Item #3.c connects Items #3.a and #3.b. Item #3.c allows Item #3.a and #3.b to be angled as desired, and then locks into place maintaining that desired angle. Item #3.b can be straight relative to Item #3.a, that is, with no angle. The tubes have rounded or flanged edges, Item #3.d, and, optionally, an inner-tube liner, Item #3.e.

### How the Invention Works

The key to the functioning of the proposed invention is that, absent the use of the proposed device, the penis with a plaque, when it becomes erect naturally, or is induced to become erect when using an artificial erection device, such as a Vacuum Erection Device (VED), will curve in the direction that is constrained to expand by the plaque. When the proposed invention is used while the penis is erect, naturally or artificially, the curvature will be constrained or countered, that is, bend in the opposite direction of the curvature.

When used in conjunction with a VED, the proposed invention remains static, as fitted by the user, to keep the erect penis straight, or passively counter the curvature of the penis. However, when used by itself during manual modeling, the user can actively set the angle of the proposed device relative to the direction of the penis to counter the curvature of the penis. In either use, the erect penis is trained, molded, or modelled in a way such that the underlying physiological causes of the curvature are reversed.

### How to Make the Invention

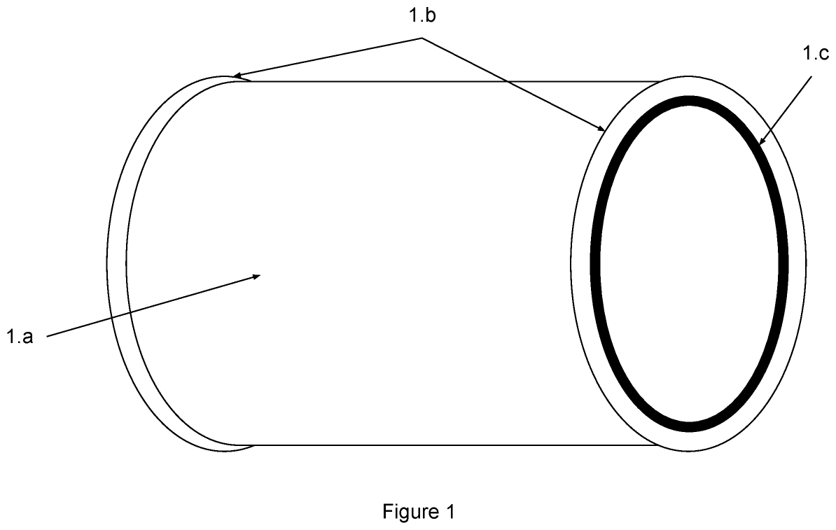

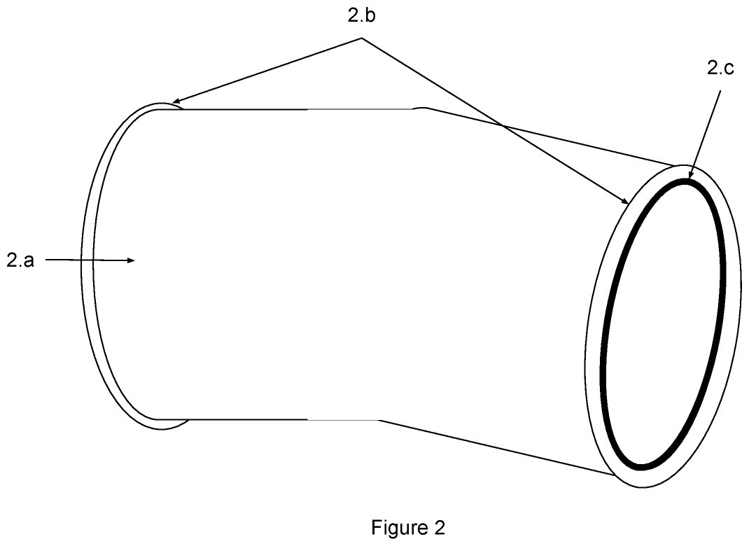

The non-bendable tubes represented by Items 1.a in FIG. 1, 2.a in FIG. 2, and 3.a and 3.b in FIG. 3 (see Drawings) can be manufactured with a hard material such as plastic, for example. They can be molded into shape. The tubes should have a rounded or flanged edge as to not injure the erect penis as it is constrained by the tubes. The latching mechanism, Item 3.c in FIG. 3, allows for a desired angle to be set and then locked into place to maintain that angle. Optionally, an inner-tube liner can be used that can be manufactured using a soft material such as felt, represented by Items 1.c in FIG. 1, 2.c in FIG. 2, and 3.e in FIG. 3.

As indicated previously with alternate versions of the proposed invention, the proposed invention can take the form of one complete component (Versions 1 and 2) as in FIGS. 1 and 2, or as multiple (nominally 2) sections (Version 3), as in FIG. 3.

### How to Use the Invention

The proposed invention is able to reverse the curvature of an erect penis suffering from, but not limited to, Peyronie's Disease by constraining or countering the curvature. Therefore, the penis is required to be erect during the use of the proposed invention. The proposed invention can be used 1) during an naturally occurring erection, either 1a) in a static position, or 1b) for performing manual modeling, or 2) when induced to become erect through the use of artificial erection devices such as Vacuum Erection Devices (VEDs), in a static position. In the latter use (2), before using the VED, the flaccid penis is inserted within the proposed invention, and then the enclosed penis is inserted into the VED. The penis will gradually become erect when using the VED, and the curvature of the penis will be constrained or countered by the proposed invention.

What is claimed is the following:

1. A penile straightening device comprising:
a. a non-bendable tube, and
b. rounded or flanged tube edges, and
c. optional inner-tube liner,
whereby said non-bendable tube encompasses an erect penis exhibiting curvature, inducing the penis to straighten passively, or to allow the wearer to actively bend the penis in the opposite direction of the curvature, and said rounded or flanged tube edges and optional inner-tube liner protect the penis from injury.

2. A penile straightening device as in claim 1, wherein said non-bendable tube is curved to counter the curvature of an erect penis.

3. A penile straightening device comprising:
a. 2 or more non-bendable tubes, and
b. 1 or more latching mechanisms, and
c. rounded or flanged tube edges, and
d. optional inner-tube liner,
whereby said non-bendable tubes encompass an erect penis exhibiting curvature inducing the penis to straighten, or to passively or actively bend the penis in the opposite direction of the curvature, and said rounded or flanged tube edges and optional inner-tube liner protect the penis from injury, and said latching mechanisms allow the non-bendable tubes to be configured at desired angles and locked into place.
